(19) United States
(12) Patent Application Publication
(10) Pub. No.: US 2004/0157770 A1
(43) Pub. Date: Aug. 12, 2004

(54) Method of inhibiting angiogenesis and neuroblastoma growth with alpha and beta isoforms of neu differentiation factor (NDF alpha and NDF beta)

(75) Inventors: George H. DeVries, Glen Ellyn, IL (US); Susan E. Crawford, Burr Ridge, IL (US)

Correspondence Address: BLANK ROME LLP, 600 NEW HAMPSHIRE AVENUE, N.W., WASHINGTON, DC, 20037, US

(73) Assignee: The United States of America as represented by the Department of Veterans Affairs, Washington, DC (US)

(21) Appl. No.: 10/360,720

(22) Filed: Feb. 10, 2003

(51) Int. Cl. A61K 38/18

(52) U.S. Cl. 514/12

(57) ABSTRACT

The invention relates to the inhibition of angiogenesis and neuroblastoma growth. In particular, the invention relates to the treatment of angiogenesis dependent and angiogenesis associated diseases, such as neural crest-derived tumors, utilizing the alpha and beta isoforms of neu differentiation factor (NDF) which have the following effects: 1) prevention of blood vessel formation, 2) induction of differentiation of neuroblastoma cells, which prevents proliferation and stops tumor growth, and 3) induction of programmed cell death (apoptosis) in neuroblastoma cells, which further inhibits tumor growth.

### FIELD OF THE INVENTION

The invention relates generally to the inhibition of angiogenesis and neuroblastoma growth. More particularly, the invention relates to the treatment of angiogenesis dependent and angiogenesis associated diseases, such as neural crest-derived tumors, utilizing the alpha and beta isoforms of neu differentiation factor (NDF) which have the following effects: 1) prevention of blood vessel formation, 2) induction of differentiation of neuroblastoma cells, which prevents proliferation and stops tumor growth, and 3) induction of programmed cell death (apoptosis) in neuroblastoma cells, which further inhibits tumor growth.

### BACKGROUND OF THE INVENTION

Angiogenesis is defined as the growth of new blood vessels out of pre-existing ones and plays a key role in a number of human diseases including malignant tumors, rheumatoid arthritis, retinal and choroidal neovascularization and some skin diseases.

Under normal physiological conditions, human and animals undergo angiogenesis only in very specific restricted situations. For example, angiogenesis is normally observed in wound healing, fetal and embryonal development, and formation of the corpus luteum, endometrium and placenta.

Angiogenesis is controlled through a highly regulated system of angiogenic stimulators and inhibitors. The control of angiogenesis has been found to be altered in certain disease states and, in many cases, pathological damage associated with the diseases is related to uncontrolled angiogensis. Both controlled and uncontrolled angiogenesis are thought to proceed in a similar manner. Endothelial cells and pericytes, surrounded by a basement membrane, form capillary blood vessels. Angiogenesis begins with the erosion of the basement membrane by enzymes released by endothelial cells and leukocytes. Endothelial cells, lining the lumen of blood vessels, then protrude through the basement membrane. Angiogenic stimulants induce the endothelial cells to migrate through the eroded basement membrane. The migrating cells form a “sprout” off the parent blood vessel where the endothelial cells undergo mitosis and proliferate. The endothelial sprouts merge with each other to form capillary loops, creating a new blood vessel.

Persistent, unregulated angiogenesis occurs in a multiplicity of disease states, tumor metastases, and abnormal growth of endothelial cells. The diverse pathological disease states in which unregulated angiogenesis is present have been grouped together as angiogenic-dependent or angiogenic-associated diseases.

The concept of tumor growth and metastasis as being angiogenesis depended, and thus therapeutic intervention being possible via angiogenic inhibition, was proposed by Judah Folkmann in 1971; and in 1976, Gullino demonstrated that cells in precancerous tissue acquire angiogenic capacity in their development to cancerous cells (Carmeliet and Jain, 2000).

To stimulate angiogenesis, tumors upregulate their production of a variety of angiogenic factors, including the fibroblast growth factor (FGF and BFGF) and vascular endothelial cell growth factor/vascular permeability factor (VEGF/VPF). However, many malignant tumors also generate inhibitors of angiogenesis, including angiostatin and thrombospordin. Several other endogenous inhibitors of angiogenesis have been identified, although not all are associated with the presence of a tumor. These include, platelet factor 4 interferon-alpha, interferon-inducible protein 10, and the 16 kDa N-terminal fragment of prolactin. Table 1 shows the role of angiogenesis in neoplasms and other diseases.

TABLE 1
Angiogenesis in neoplasma and diseases

| | Process characterized by abnormal or vascular malfunction | | | |
|---|---|---|---|---|
| | | | | Increased |
| | | | Abnormal | Vascularization and/or |
| Organ | Increases vascularization | Insufficient Vascularization | remodeling | permeability |
| Blood vessels | Atherosclerosis, hemangioma, | | Vascular | |
| | hamangioendothelioma | | Malformations | |
| Skin mucosa | Warts, pyogenic granulomas, | Decubitus or stasis ulcers, | Psoriasis, | |
| | hair growth, Kaposis sarcoma, | gastrointestinal ulcers | | |
| | scar | | | |
| Uterus, ovary, | Dysfunctional uterine bleeding, | Placental insufficiency | Pre-eclampsia | |
| placenta | follicular cysts, ovarian tumors | | | |
| Peritoneum, pleura | | | | Respiratory distress, |
| | | | | ascites, peritoneal |
| | | | | Sclerosis, adhesion |
| | | | | formation, metastatic |
| | | | | spreading |
| Heart, skeletal | Work overload | Ischemic heart and limb disease | | |
| muscle | | | | |
| Adipose tissue | Obesity | | | |
| Bone, joints | Rheumatoid arthritis, synovitis, | Aseptic necrosis, impaired | | |
| | bone and cartilage destruction, | healing of fractures | | |
| | osteomyelitis, pannus growth, | | | |
| Liver, kidney, ear | Inflammatory and infectioius | Pulmonary and systemic | Pulmonary | |
| and other epithelia | processes, asthma, nasal polyps, | hypertension | Hypertension, | |
| | transplantation, liver | | diabetes | |
| | regeneration, | | | |
| Brain, nerves, eye | Retinopathy of prematurity, | Stroke, vascular dementia, | | |
| | diabetic retinopathy, choroidal | Alzheimer's disease | | |
| | and other | | | |
| Endocine organs | Thyroiditis, thyroid enlargement, | Thyroid pseulocyst | | |
| Lymph vessels | Tumor metastasis, | Lymphedema | | |

Excessive or insufficient vascular growth contributes to many other non-neoplastic diseases. Hypoxia occurs in atherosclerotic plaques, diabetes, and Alzheimer's disease when cells become situated too far from blood vessels, and this provides a stimulus for angiogenesis. Hypoxia-driven angiogenesis can cause blindness in premature newborns and diabetic patients and hemorrhagic rupture of atherosclerotic plaques. Inflammatory disorders can also stimulate angiogenesis, and angiogenesis is thought to contribute to the excess accumulation of body fat in obese individuals.

Thus, it is clear that angiogenesis plays a major role in the metastasis of cancer. If this angiogenic activity could be repressed or eliminated, then the tumor, although present, would not grow. In the disease state, prevention of angiogenesis could avert the damage caused by the invasion of the new microvascular system. Therapies directed at control of the angiogenic processes could lead to the abrogation or mitigation of these diseases.

Angiogenesis has been associated with a number of different types of cancer, including solid tumors, blood-borne tumors, and neural crest-derived tumors, that include neuroblastoma, ganglio-neuroblastroma, retinoblastoma, primitive neuroectodermal tumors (PNET), neuroflibrosarcomas and others.

Neuroblastoma is a cancer of the sympathetic nervous system and is a solid, malignant tumor, which manifests as a lump or mass in the abdomen or around the spinal cord in the chest, neck or pelvis. Neuroblastoma tumors are composed of neuroblasts, neuropil, and Schwann cell-like stroma, and it appears that there is an inverse relationship between the number of Schwann cells within tumor and the prognosis for tumor growth. Neuroblastoma is the most common childhood solid tumor arising outside of the brain and occurs in approximately 1 in 100,000 live births in the U.S., leading to 550 new cases each year. Two third of neuroblastoma cases in children younger than 1 year of age, even those with advanced disease but favorable disease characteristics, have a high likelihood of long-term disease-free survival. Overall, neuroblastoma claims the lives of up to 50 percent of those children diagnosed with the disease after the age of 1.5 years, probably due in part to the fact that a high proportion of cases have metastasized by the time they are diagnosed.

Presently, conventional cancer chemotherapy is seen as highly inadequate because of a lack of specificity to cancer cells, with the result that many normal cells are destroyed, causing severe adverse effects. Current treatment for neuroblastoma depends on the stage of the neuroblastoma. There are a number of staging systems used for this disease, but the two described by the National Cancer Institute, based on the Children's Cancer Group (CCG), St. Jude and the Pediatric Oncology Group (POG) staging systems, are generally the most accepted. The treatment options include surgery, radiotherapy, chemotherapy (daunorubicin, cyclophosphamide, carboplatin and etoposide) and bone marrow transplantation (autologous bone marrow transplatation following aggressive chemotherapy). The chemotherapy options are all cytotoxic drugs, and are associated with serious side effects. The treatment options available for neuroblastoma are similar to those for other types of cancer therapy, where an angiogenesis inhibitor could either replace or be used in conjunction with conventional treatments, and perhaps enable smaller doses of radio- and chemotherapy to be used, or perhaps obviate the need for surgery.

The above methods for treatment of neuroblastoma tumors lack adequate potency or are too toxic for practical use. Thus, methods and compositions are needed that are easily administered and capable of inhibiting angiogenesis.

### SUMMARY OF THE INVENTION

The present invention relates the use of the alpha and beta isoforms of neu differentiation factor (NDF-α and NDF-β) as anti-cancer agents, in the first instance in the treatment of angiogenic dependent diseases and additionally as a treatment for neuroblastoma, based on two different actions of the same compound (inhibition of proliferation via promotion of differentiation and induction of programmed cell death).

It was found that there is inverse relationship between the number of Schwann cells within a tumor and a favorable prognosis for tumor growth. The hypothesis has been developed that this relationship is the result of the secretion of certain factors by the Schwann cells that lead to the more favorable prognosis. To date, these factors have not yet been identified. We have discovered that Schwann cells contain and secrete both the alpha (a) and beta (>) isoforms of NDF (Raabe et al., J. Neurosci. Res., 46:263-270, 1996) and that the secreted NDF-α and NDF-β inhibit tumor cell proliferation and angiogenesis, thus preventing tumor growth. Neu differentiation factor (NDF) is a member of the family of ligands for tyrosine kinase receptors known as the erb B family consisting of four distinct cell surface receptors.

Neu differentiation factors act through activation of the tyrosine kinase receptors coded by the erbB family of genes. One member of this family is erbB-1, coding for the epidermal growth factor receptor (EGFR). This receptor is known to be overexpressed in a number of tumors and involved in the proliferation of these cells. The neu differentiation factor binds erbB-3 and erbB-4 receptors, and can also activate the erbB-2 receptor, and has been shown to activate these receptors in a variety of tumor cell lines. The variable activation of various erb B receptors is responsible for the variety of biological effects exhibited by neu differentiation factors.

NDF-α and NDF-β are tyrosine kinase activators, and the signaling pathways in which they are involved play key roles in a variety of normal cells including the growth of epithelial cells, angiogenesis, the proliferation of connective tissue cells, and the regeneration of tissue during wound healing. There is also evidence of cross-talk between heterologous signaling pathways, which could mean that NDF-α and NDF-β have the potential to be effective in preventing proliferation on cancers other than neuroblastoma (Moghal and Sternberg, Curr. Opin. Cell Biol., 11(2):190-196, 1999).

“NDF” is a 44-kilodalton polypeptide originally isolated from rat fibroblasts which has been shown to induce the growth or differentiation of epithelial cells (Peles et al., Cell, 69:205-216, 1992). Both this heat-stable rodent protein and its human homologue, called heregulin, are secreted proteins and related to a class of growth factors called neuregulins.

Neuregulins are a class of growth factors that activate tyrosine kinase receptors that are collectively referred to as ErbB receptors. The neuregulins were independently cloned by a number of different investigators who gave the molecules a variety of monikers including ARIA (acetylcholine receptor inducing activity), heregulins (human form of the growth factor), glial growth factor II (a pituitary factor used as a crude mitogenic extract for glial cells) and NDF (neu differentiation factor-a growth factor that activated the ErbB-2 receptor or the neu receptor and caused differentiation of certain cells in culture). The term “neuregulin” was devised to include all molecules that activate any of the four distinct Erb-β receptors.

Neuregulins are synthesized initially as membrane bound molecules and subsequently released by proteolysis at a juxtamembrane site. Functionally, the molecule is divided into the following domains from the carboxylterminal end: a cytoplasmic domain, an EGF domain which is the active part of the molecule that combines with the erb-β receptors, a variable glycosylation domain, an Ig domain, and a hydrophobic leader sequence domain at the amino terminal.

There are a number of functions, other than those described herein that have been discovered for the neuregulins. They can be divided into the following four categories: (a) differentiation of mammary epithelial cells and oligodendrocytes, (b) stimulation of mitosis in Schwann cells, (c) prevention of apoptosis in Schwann cells, oligodendrocytes and astrocytes, and (d) clustering of acetylcholine receptors (ARIA). To date, no other biological activities have been reported.

Neuroblastoma tumors are one of the most common solid malignancies in children. Although therapeutic advances have been made in to other childhood cancers, the mortality rate for neuroblastoma has been relatively stable at 40-50%. This tumor is composed of primitive neuroblasts, neuropil, and Schwannian stroma. The Schwann cell component is considered reactive and consequently, a higher composition of Schwann cells within a tumor correlates with a better prognosis. The factors secreted by Schwann cells that contribute to the more favorable prognosis have not been clearly identified. We believe that NDF-α and NDF-β are the major factors responsible for this anti-tumor effect.

The invention is based on the discovery that NDF-β and NDF-α (a Schwann cell-derived factor) can induce differentiation in neuroblastoma tumor cells. The data results suggest that one of the reasons that the presence of Schwann cells within neuroblastoma tumors leads to a more favorable prognosis is that these cells are producing NDF-α and NDF-β. Both isoforms of the NDF can then act in a paracrine fashion on the tumor cells to induce their differentiation. Additionally, NDF-α or NDF-β can suppress endothelial cell proliferation and block the blood supply to the tumor, thus, reducing its ability to grow.

We have demonstrated that NDF-α or NDF-P can induce differentiation in neuroblastoma tumor cells, presumably through its action on the erbB-3 and erbB-4 receptors. NDF-α and NDF-β appear to have antitumor properties in treating neuroblastoma via their ability to inhibit angiogenesis, thereby blocking the supply of blood to tumors and metastases. In addition, these growth factors can induce cell differentiation and induces apoptosis, which stops the proliferation of neuroblastoma cells.

Since in-vivo and in-vitro studies on the subject technology have demonstrated anti-angiogenic as well as anti-proliferative properties in suppressing neuroblastoma neoplasm growth, it is possible that the subject technology proteins may have biological activity against other types of angiogenic dependent disease such as rheumatoid arthritis, diabetic retinopathy, and atherosclerosis and others (see Table 1). Additionally, identification of these naturally occurring factors may lead to development of therapies targeting the protein in cases of insufficient angiogenesis.

We have also demonstrated in vitro the ability of NDF-α and NDF-β to cause the differentiation of cells obtained from neuroblastoma cells, an action that would prevent proliferation. In addition we have directly demonstrated that NDF can inhibit the proliferation of neuroblastoma cells. Moreover we have shown that NDF can cause activation of programmed cell death in the neuroblastoma cells.

The present invention also relates to methods of using NDF-cc and NDF-P for treating angiogenesis-related diseases, particularly angiogenesis-dependent tumors. The method unexpectedly provides the medically important results of inhibiting angiogenesis thereby provide a reduction of tumor mass.

It is yet another object of the invention to provide NDF-α and NDF-β that are Schwann cell-derived factors that induce differentiation and apoptosis in neuroblastoma tumor cells in vitro.

It is a further object of the present invention to provide a method of treating diseases and processes that are mediated by angiogenesis.

It is yet another object of the present invention to provide a method for treating diseases and processes that are mediated by angiogenesis including, but not limited to, hemangioma, solid tumors, leukemia, metastasis, telangiectasia, psoriasis, scleroderma, pyogenic granuloma, myocardial angiogenesis, plaque neovascularization, corornay collaterials, cerebral collaterals, arteriovenous malformations, ischemic limb angiogensis, corneal diseases, rubeosis, neovascular glaucoma, diabetic retinopathy, retrolental fibroplasias, arthritis, diabetic neovascularization, macular degeneration, wound healing, peptic ulcer, fractures, keloids, vasculogenesis, hematopoiesis, ovulation, menstruation, and placentation.

It is yet another object of the present invention to provide a method for treating or repressing the growth of a cancer.

It is another object of the invention to provide a method for inhibiting angiogenesis.

These and other objects, features, and advantages of the present invention will become apparent after a review of the following detailed description of the disclosed embodiments and the appended claims.

### BRIEF DESCRIPTION OF THE DRAWINGS

FIG. 1 shows that NDF, found in the conditioned media of Schwann cells and whole nerve, inhibits angiogenesis via the inhibition of endothelial cell migration.

FIG. 2 shows that NDF promotes the differentiation of neuroblastoma cells.

### DETAILED DESCRIPTION OF THE PREFERRED EMBODIMENTS

We have discovered that both neu differentiation factor-alpha (NDF-α) and neu differentiation factor-beta (NDF-β) derived from Schwann cells have the ability to inhibit angiogenesis and to block blood vessel growth when added to proliferating endothelial cells in vitro and able to block neovascularization from the corneal limbus in vivo. NDF-β and NDF-α have two potent biological activities. They can act as a naturally occurring inhibitor of angiogenesis to block the blood supply to the tumor; they also can induce differentiation of tumor cells. These two actions have been shown to be important in curtailing or stabilizing tumor growth.

The present invention encompasses the use of NDF-β and NDF-α and active regions of these molecules as a potent inhibitor of angiogenesis, and prevention of proliferation of tumor cells.

A method for the production of NDF-β or NDF-α is well known in the art. Examples of suitable methods are disclosed is Holmes et al., Science, 256:1205, 1992, which is incorporated here in by reference in its entirety. Basically, the growth factor is made by recombinant DNA procedures in E. Coli with the active domain of the molecule, namely, amino acids 177-244 being expressed. The NDF-B and NDF-α used in these studies was purchased from a commercial supplier (R&D Systems, Minneapolis, Minn.) provided with detailed specification sheet form for preparation of the molecule. The purity is greater than 97%. Activity and how the material was prepared and stored was provided with the detailed specification sheet.

NDF-β can be administered intraperitonally, intravenously or as an addition to a topical cream in concentrations sufficient to achieve nanomolar levels in tissue.

NDF-β was tested in two common bioassays for its angiogenic activity using an in vitro endothelial cell migration assay and an in vivo rat cornea neovascularization assay. Both assays revealed that NDF-β is a potent inhibitor of endothelial cell migration and proliferation. These data suggest that supplying this molecule either systemically or topically may suppress unwanted neovascularization associated with angiogenic-dependent diseases such as tumors, rheumatoid arthritis, diabetic retinopathy, and atherosclerosis. Since this molecule is secreted from normal cells (Schwann cells) and whole nerve preparations, the toxicity from any treatment regimens should be minimal.

The present invention also encompasses the use of purified NDF-α or NDF-β as a Schwann cell-derived factor that induces differentiation in neuroblastoma tumor cells in vitro.

In vitro treatment of neuroblastoma cells (America Tissue Type and Culture, ATCC, SK− N-BE (2) and SK-N—SH; human tumor cells from two different patients) with either NDF-α or NDF-β, induced differentiation suggesting that supplying purified NDF-α or NDF-β will be an effective method to induce these tumors to differentiate and therefore grow more slowly. Neuroblastomas are one of the most common solid malignancies in children. Despite advances in therapy, it still claims the lives of up to 50% of those children diagnosed after the age of 1.5 years.

The following examples are given to illustrate the present invention. It should be understood that the invention is not limited to the specific conditions or details described in these examples.

### EXAMPLE 1

### In Vitro and In Vivo Anti-Angiogenic Activity of NDF

Experiments were conducted to determine if NDF-β had angiogenic activity using two commonly used bioassays: inhibition of endothelial cell migration and the in vivo rat corneal neovascularization assay.

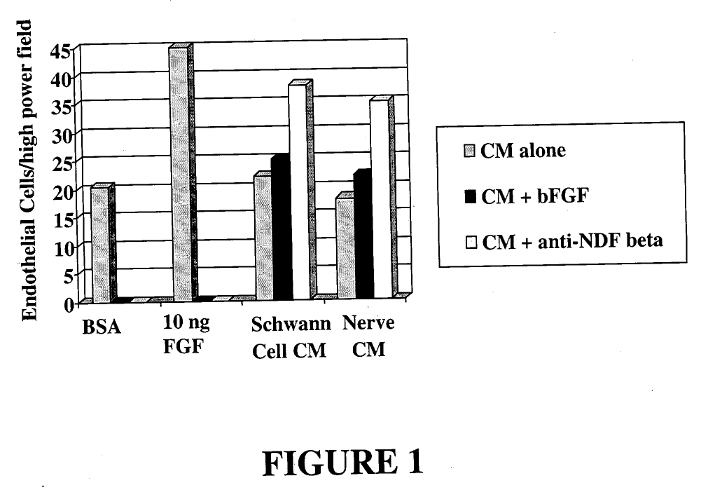

To establish that human Schwann cells within a tumor secrete NDF, which is anti-angiogenic, Schwann cells were cultured from the neuroblastoma tumors and cultured for 48 hours to condition the medium. The conditioned medium was analyzed for anti-angiogenic activity by studying the ability of the conditioned media to inhibit endothelial cell migration. To determine if this inhibitory activity was due to NDF-β, an antibody that neutralizes the biological activity of this protein, was mixed with media followed by testing of the antibody treated media for its ability to inhibit endothelial cell migration. Schwann cells are a component of the normal whole nerve; therefore, incubation of minced peripheral nerve containing Schwann cells should generate a conditioned media, which is anti-angiogenic due to the secretion of anti-angiogenic factor by the Schwann cells. To test this possibility, whole human peripheral nerves were minced and incubated for 48 hours in tissue culture medium. The whole nerve conditioned media was then collected and tested under the same conditions. Capillary endothelial cells were cultured and seeded onto a nylon membrane in a Boyden chamber under the following conditions: medium plus bovine serum albumin only (FIG. 1, gray bar labeled BSA) which is the background migration of the endothelial cells; medium plus bFGF (FIG. 1, gray bar labeled bFGF) which is the induced migration of the endothelial cells, conditioned medium only (FIG. 1, gray bars in the three bar clusters); conditioned medium plus bFGF (FIG. 1, black bars) and conditioned medium plus neutralizing antibody to NDF-β (FIG. 1, white bars). At the end of the assay the filters are examined by microscopy under a high power field to determine the number of endothelial cells, which have migrated through the filter. The results of a typical experiment are shown in FIG. 1.

This in vitro endothelial migration assay showed that the conditioned media (secretions of Schwann cells) were anti-angiogenic since the conditioned media blocked the inducing angiogenic activity of bFGF (FIG. 1, black bars) (in the uninhibited condition the black bars would have been equivalent in height to the bar labeled “10 ng FGF”). The conditioned media which was treated with neutralizing antibody to NDF (FIG. 1, white bars) showed release of the inhibitory activity strongly suggesting that NDF-β is one of the major inhibitors of angiogenesis secreted by normal Schwann cells and whole nerve.

Conditioned media was collected and the media tested in a Western blot using an antibody to NDF-β. The Western blot revealed a discrete protein band at the correct molecular weight similar to what we observed in the conditioned media of rat Schwann cells.

Taken together, these data suggest that Schwann cells and Schwann cells contained in whole nerves secrete sufficient levels of NDF to inhibit angiogenesis as shown by the inhibition of endothelial cell migration.

To confirm the inhibitory activity of NDF-β in vivo, this protein was tested in a rat-corneal-neovascularization-assay. In this assay, a hydron pellet containing the test substance is implanted into the vascular limbus of the rat cornea. The hydron pellets are made using a solution that is 12% (w/v) Hydron (Interferon Sciences, New Brunswick, N.J.) in 96% Ethanol. Test substance (25 μl in sterile phosphate buffered saline) is mixed with 25 μl of 12% Hydron. A small drop of this mixture is placed on the top of Teflon® pegs (2/5-3 mm in diameter) in the sterile environment of a laminar flow hood. As the solution evaporates, flat pellets form. Conditioned media is used at 200 μg/ml, the inducer bFGF is used at 100 ng/ml. The final volume of the test pellet implanted into each cornea is approximate 5 μl. Seven days later, animals are anesthetized and perfused with colloidal carbon to fill the blood vessels so that any new vessel growth in the cornea will be highlighted. The results of this assay are shown in Table 2.

TABLE 2
NDF Inhibits Blood Vessel Formation In vivo
(Rat Corneal Neovascularization Assay)

| Treatment | *Results |
|---|---|
| BSA alone | 0/2 |
| Basic FGF alone | 2/2 |
| Basic FGF + NDF Beta (1 nM) | 0/2 |

*Number of positive corneas/total corneas tested

Vigorous brush-like vessel growth toward the bFGF-containing pellet was noted in the bFGF treated corneas and was scored as a positive angiogenic response. When NDF-β was mixed with a known angiogenesis inducer (bFGF), the mixed pellets containing the NDF showed no neovascularization from the limbus of the rat cornea, demonstrating the ability of NDF to block angiogenesis. This assay confirmed that NDF was able to block angiogenesis in vivo as revealed by blocking neovascularization from the corneal limbus, even in the presence of a potent angiogenesis inducer, basic FGF.

In summary, our data suggest that NDF from a variety of sources, including the purified protein, inhibits corneal neovascularization in normal rat. Schwann cell conditioned media, human neuroblastoma Schwann cells, or whole nerve preparations, are potent inhibitors of angiogenesis and may prove to be beneficial in suppressing or stabilizing neuroblastoma tumor growth or treating other angiogenic dependent disease processes

### EXAMPLE 2

### NDF Induces Differentiation in Neuroblastoma Cells

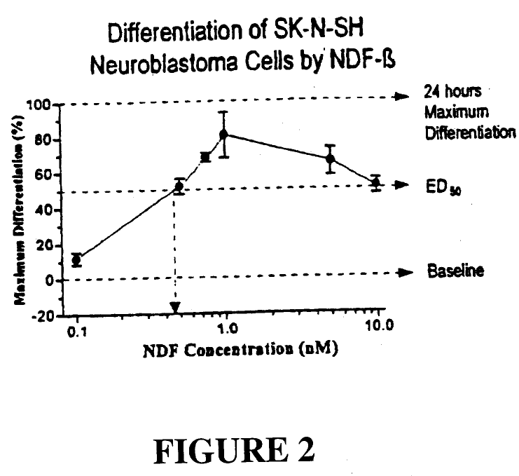

In vitro experiments were conducted to determine the ability of NDF to induce the differentiation of neuroblastoma cells using cell lines derived from neuroblastoma tumors. The two neuroblastoma cells lines (SK-N—SH and SK-N-BE) are described in Barnes et al., In Vitro, 17(7):619-631, 1981, and were obtained from the American Tissue Type and Culture (ATCC). Both cell lines were maintained in culture with DMEM containing 10% fetal bovine serum (Flow Laboratories, McLean, Va.) at 37° C. and 5% CO₂. Cells (1.25×10⁴) were resuspended, and 1 ml/well was used to seed 24 well plates. Twenty-four hours later, NDF in serum free medium was added to triplicate wells at concentrations of 0, 0.1, 0.5, 0.75, 1.0, or 10.0 nM, and cells incubated for an additional 24 hours. The percentage of differentiated cells was determined by counting the total number of cells in three non-overlapping 1 mm² areas per well. A cell was considered differentiated if it possessed neurite outgrowth greater than 50 microns in length. The results of a typical dose response curve are shown in FIG. 2.

As shown in FIG. 2, it is evident that NDF is a strong inducer of the differentiation of neuroblastoma cells with an ED-50 of approximately 0.45 nM. When an antibody to NDF was added in these experiments, no effect on neuroblastoma differentiation was noted, indicating that the biological activity was, indeed, due to NDF. These experiments have been carried out with both the alpha and beta isoforms of NDF.

### EXAMPLE 3

### NDF Inhibits Proliferation in Neuroblastoma Cells

In order to demonstrate that proliferation of neuroblastoma cells is inhibited as a consequence of increased differentiation by NDF, the following experiment was carried out. The SK-N-BE neuroblastoma cells (5,000) were placed in wells of a tissue culture plate and allowed to incubate in a media of DMEM plus 5% fetal calf serum for eight days in the presence or absence of 50 ng/ml NDF. The cells were then analyzed in a standard DNA-fluorescence based proliferation assay (Cyquant™, Molecular Probes, Eugene, Oreg.). The results are shown in Table 3, which shows strong inhibition of proliferation induced by either the α or β isoforms of NDF.

TABLE 3
NDF inhibits Neuroblastoma Cell Proliferation

| Condition | DNA Fluorescence at 530 nm | % Inhibition |
|---|---|---|
| Control | 2664 ± 318 | 0% |
| NDF-α (50 ng/ml) | 1715 ± 77 | 46% |
| NDF-β (50 ng/ml) | 1559 ± 53 | 42% |

### EXAMPLE 4

### NDF Induces Apoptosis in Neuroblastoma Cells

In addition to inhibition of proliferation, we evaluated the ability of NDF to induce apoptosis. Neuroblastoma cells (20,000) were incubated in DMEM containing 1% fetal calf serum in the presence or absence of the indicated concentrations of NDF. After 48 hours, the cells were stained with Hoechst Dye (33342) and visualized on a Zeiss Fluorescent microscope. The labeled nuclei were then scored as apoptotic if the nuclei were fragmented. The percentage of apoptotic nuclei was calculated after evaluating at least 100 cells as. The result is depicted in Table 4, which shows 5 to 6 fold increase in apoptosis induced by either the α or β isoforms of NDF relative to the low percentage of apoptotic cells noted in control cells in serum containing medium. Combined with the other anti-tumor effects of NDF, this activity could make a potent contribution to inhibiting tumor growth.

TABLE 4
NDF Induces Apoptosis in Neuroblastoma Cells

| Treatment | % Apoptosis | Total Cells evaluated |
|---|---|---|
| Control | 9 | 100 |
| NDF-α (20 ng/ml) | 60 | 203 |
| NDF-α (40 ng/ml) | 56.7 | 428 |
| NDF-β (27 ng/ml) | 45.0 | 376 |
| NDF-β (54 ng/ml) | 49.5 | 111 |

Treating Retinoblastoma

The test results presented above with respect to NDF indicate its usefulness as an agent that could be used both as an anti-angiogenic in cases of excessive angiogenesis and as a possible target for promoting angiogenesis when angiogenesis is insufficient.

As can be seen from the results, NDF or the active region of the protein, or the gene encoding either of these could be developed into a treatment regimen for neuroblastoma tumors or other angiogenic-dependent diseases.

NDF as an anti-tumor agent may have several advantages over traditional chemotherapeutic therapies. NDF is secreted by normal cells and, therefore, it is less likely to exert a toxic effect on neighboring cells. Moreover, NDF could be delivered to areas in which neuroblastoma or other solid tumors are growing in order to effectively control tumor growth. In addition, the dual action of NDF as an inhibitor of angiogenesis and as a tumor cell-differentiating factor may result in enhanced therapeutic potency in suppressive tumor growth.

The invention has been disclosed broadly and illustrated in reference to representative embodiments described above. Those skilled in the art will recognize that various modifications can be made to the present invention without departing from the spirit and scope thereof.

What is claimed is:

1. A method of inhibiting angiogenesis comprising the step of administering an angiogenesis-inhibiting amount of the a or P isoform of neu differentiation factor (NDF).

2. The method of claim 1, wherein said NDFα and β are Schwann cell-derived anti-angiogenic factors.

3. The method of claim 2, wherein said NDFα or β is an erbB-2, erbB-3, or erbB-4 tyrosine kinase receptor activator.

4. The method of claim 1, wherein said NDF is administered in an amount sufficient to achieve a tissue concentration of 10 ng/ml.

5. The method of claim 1, wherein said NDF is administered in a purified form.

6. The method of claim 1, wherein said NDF is implanted into the avascular limbus of the cornea of said subject.

7. The method of claim 1, wherein said NDF is implanted in a form of a pellet.

8. The method claim 1, for treating diseases selected from the group consisting of cells, which are of neural crest origin consisting of neural-derived neuroblastoma, ganglioneuroblastoma, retinoblastoma, primitive neuroectodermal tumors (PNET) neurofibrosarcomas, neurofibromas, and malignant peripheral nerve sheath tumors.

9. A method of treating neuroblastoma in a subject comprising the step of administering to said subject an angiogenesis-inhibiting amount of NDF in purified form.

10. The method of claim 9, wherein the alpha and beta isoforms of NDF are Schwann cell derived anti-angiogenic factors.

11. The method of claim 9, wherein said alpha and beta isoforms of NDF are erbB2, erbB-3 and erbB-4 tyrosine kinase receptor activators.

12. The method of claim 9 wherein said alpha and beta isoforms of NDF are initiators of programmed cell death in tumor cells.

13. A method of inducing differentiation of neuroblastoma cells comprising contacting said cells with neu differentiation factor (NDF).

14. The method of claim 13, wherein said NDF is Schwann cell-derived.

15. The method of claim 13, wherein said NDF is selected from the group consisting of NDFα and NDFβ.

16. The method of claim 13, wherein said NDFα or β is an erbB-2, erbB-3 and erbB-4 tyrosine kinase receptor activator.

17. The method of claim 13, wherein said NDF is administered in an amount sufficient to achieve a tissue concentration of about 10 ng/ml.

18. The method of claim 13, wherein said NDF is administered in a purified form.

19. The method of claim 5, wherein said NDF is implanted into the avascular limbus of the cornea of said subject.

20. The method of claim 6, wherein said NDF is implanted in a form of a pellet.

21. A method of inducing apoptosis comprising the step of administering an apoptosis-inducing amount of the a or P isoform of neu differentiation factor (NDF).

22. The method of claim 21, wherein said NDFα and β are Schwann cell-derived anti-angiogenic factors.

23. The method of claim 22, wherein said NDFα or β is an erbB-2, erbB-3, or erbB-4 tyrosine kinase receptor activator.

24. The method of claim 21, wherein said NDF is administered in an amount sufficient to achieve a tissue concentration of 10 ng/ml.

25. The method of claim 21, wherein said NDF is administered in a purified form.

26. The method of claim 21, wherein said NDF is implanted into the avascular limbus of the cornea of said subject.

27. The method of claim 21, wherein said NDF is implanted in a form of a pellet.

28. The method claim 21, for treating diseases selected from the group consisting of cells, which are of neural crest origin consisting of neural-derived neuroblastoma, ganglioneuroblastoma, retinoblastoma, primitive neuroectodermal tumors (PNET) neurofibrosarcomas, neurofibromas, and malignant peripheral nerve sheath tumors.

29. A method of inducing differentiation comprising the step of administering an differentiation-inducing amount of the α or β isoform of neu differentiation factor (NDF).

30. The method of claim 29, wherein said NDFα and β are Schwann cell-derived anti-angiogenic factors.

31. The method of claim 30, wherein said NDFα or β is an erbB-2, erbB-3, or erbB-4 tyrosine kinase receptor activator.

32. The method of claim 29, wherein said NDF is administered in an amount sufficient to achieve a tissue concentration of 10 ng/ml.

33. The method of claim 29, wherein said NDF is administered in a purified form.

34. The method of claim 29, wherein said NDF is implanted into the avascular limbus of the cornea of said subject.

35. The method of claim 29, wherein said NDF is implanted in a form of a pellet.

36. The method claim 29, for treating diseases selected from the group consisting of cells, which are of neural crest origin consisting of neural-derived neuroblastoma, ganglioneuroblastoma, retinoblastoma, primitive neuroectodermal tumors (PNET) neurofibrosarcomas, neurofibromas, and malignant peripheral nerve sheath tumors.
